(19) United States
(12) Patent Application Publication
(10) Pub. No.: US 2017/0336344 A1
(43) Pub. Date: Nov. 23, 2017

(54) GAS CONCENTRATION DETECTION DEVICE

(71) Applicant: NGK SPARK PLUG CO., LTD., Nagoya-shi (JP)

(72) Inventors: Tomonori UEMURA, Komaki-shi (JP); Satoru ABE, Ichinomiya-shi (JP); Tetsuya ITO, North Nagoya-shi (JP); Satoshi TERAMOTO, Nisshin-shi (JP)

(73) Assignee: NGK SPARK PLUG CO., LTD., Nagoya-shi (JP)

(21) Appl. No.: 15/598,800

(22) Filed: May 18, 2017

(30) Foreign Application Priority Data
May 19, 2016 (JP) 2016-100624

(51) Int. Cl. G01N 27/41; G01N 27/407; G01N 33/22

(52) U.S. Cl. CPC G01N 27/41; G01N 33/225; G01N 27/4071; G01N 27/4073; G01N 27/4075

(57) ABSTRACT

A gas concentration detection device for detecting a gas concentration using a limiting current type gas concentration sensor. In an application voltage line set to pass through a plurality of limiting current regions for different values of gas concentration, the voltage set by the application voltage line is an identical first voltage value used when an air/fuel ratio corresponding to the gas concentration is in a predetermined first range, is an identical second voltage value different from the first voltage value used when the air/fuel ratio is in a second range adjacent to the first range, and the first voltage value and the second voltage value are switched between the first range and the second range.

### BACKGROUND OF THE INVENTION

### 1. Field of the Invention

The present invention relates to a gas concentration detection device for detecting the concentration of a specific gas contained in a gas to be measured.

### 2. Description of the Related Art

Conventionally, for example, a limiting current type air/fuel ratio sensor is known as a device for detecting the concentration (i.e., air/fuel ratio: A/F) of oxygen in an exhaust gas discharged from an engine of a vehicle.

This type of air/fuel ratio sensor has, as a sensor element, for example, a solid electrolyte and a pair of electrodes formed thereon, and is configured such that current (pump current Ip) corresponding to oxygen concentration flows by applying a voltage (application voltage Vp) between the pair of electrodes.

FIG. 4A shows output characteristics indicating the relationship between pump current Ip and application voltage Vp of the above sensor element. In the output characteristics, a flat region parallel to the voltage axis, i.e., a region (limiting current region) GD of limiting current is known in which the pump current Ip is constant. In addition, the pump current Ip in the limiting current region GD is known to increase as the oxygen concentration increases.

Therefore, conventionally, an application voltage Vp corresponding to the limiting current region GD is applied to the sensor element, and the oxygen concentration is detected from the resulting pump current Ip. That is, the oxygen concentration (i.e., air/fuel ratio) is detected by a so-called limiting current method.

In the above technique, in order to detect the air/fuel ratio accurately, it is necessary to control the application voltage Vp in a range corresponding to the limiting current region GD. Therefore, normally, using a linear function indicating a straight line, an application voltage line ID indicating the relationship between the application voltage Vp and the pump current Ip (for determining the application voltage Vp) is set, and then the application voltage Vp is determined using the application voltage line ID.

However, as shown in FIG. 4B, the output characteristics and the limiting current region GD change depending on the temperature (high-temperature side H, low-temperature side L). Therefore, in recent years, a new method for setting the application voltage line ID has been proposed (see, for example, Patent Document 1).

In this measurement method, the application voltage line ID is set using a single straight line (linear function) so as to pass through a region in which the limiting current regions GD of a plurality of output characteristics (high-temperature side H, low-temperature side L) for different temperature conditions overlap each other.

[Patent Document 1] Japanese Patent No. 4124119

### 3. Problems to be Solved by the Invention

However, the conventional technique in which the application voltage line ID is set using a single straight line merely by considering a temperature condition as described above is not always sufficient.

That is, in practice, depending on the gas atmosphere (i.e., oxygen concentration) or variation among individual sensor elements, the resistance value of the solid electrolyte thereof varies, and therefore there is a possibility that the detection accuracy of the oxygen concentration is deteriorated.

For example, even if the temperature condition is considered, depending on the oxygen concentration (for example, in a rich case where the amount of fuel is larger than in a stoichiometric state), the application voltage line ID using a single straight line can deviate from the limiting current regions GD. In such a case, even if control using the application voltage line ID is performed, the oxygen concentration cannot be accurately detected.

### SUMMARY OF THE INVENTION

It is therefore an object of the present invention to provide a gas concentration detection device capable of accurately detecting a gas concentration in the case of detecting gas concentration using a limiting current type gas concentration sensor.

The above object of the invention has been achieved by providing the following.

(1) In a first aspect, the present invention relates to a gas concentration detection device, adapted for operating a gas concentration sensor including a sensor element having: a solid electrolyte having oxygen ion conductivity; and a pair of electrodes formed on the solid electrolyte, the gas concentration detection device being configured to apply a voltage between the pair of electrodes based on an application voltage line which defines a relationship between voltage applied between the pair of electrodes and current flowing between the pair of electrodes, detect a limiting current flowing between the pair of electrodes in accordance with the voltage, and detect a gas concentration of a specific component in a gas to be measured, based on the limiting current.

In the gas concentration detection device (1), in a detection range for detecting the gas concentration, the application voltage line is set so as to pass through a plurality of limiting current regions of: respective limiting current regions for different values of the gas concentration; and a region in which respective limiting current regions for different temperature conditions of the sensor element overlap each other. The voltage set by the application voltage line is an identical first voltage value used when an air/fuel ratio corresponding to the gas concentration is in a predetermined first range, is an identical second voltage value different from the first voltage value used when the air/fuel ratio is in a second range adjacent to the first range, and the first voltage value and the second voltage value are switched between the first range and the second range.

Thus, in the first aspect, as a basic configuration, the application voltage line is set so as to pass through respective limiting current regions for different gas concentrations and to pass through a region in which respective limiting current regions for different temperature conditions of the sensor element overlap each other.

In addition, in the first aspect, in the basic configuration described above, the voltage set by the application voltage line is an identical first voltage value used when the air/fuel ratio corresponding to the gas concentration is in the predetermined first range, an identical second voltage value different from the first voltage value used when the air/fuel ratio is in the second range adjacent to the first range, and the first voltage value and the second voltage value are switched (in step form) between the first range and the second range. Therefore, it is possible to accurately detect the gas concentration (specifically, air/fuel ratio).

That is, when considering the temperature condition, for example, depending on the oxygen concentration or the like, the application voltage line using a single straight line can deviate from some limiting current regions. In this case, it might be impossible to accurately detect, for example, the oxygen concentration, even if control using this application voltage line is performed.

However, in the first aspect, the detection range for detecting the gas concentration is divided into at least two first and second ranges adjacent each other. The first voltage value which is an identical fixed value is set for the first range and the second voltage value which is an identical fixed value is set for the second range, so as to pass through limiting current regions for different gas concentrations in each range, and a region in which limiting current regions for different temperature conditions of the sensor element overlap each other. Further, the first voltage value and the second voltage value are switched between the first range and the second range, so as to prevent the application voltage line from deviating from the limiting current regions.

Therefore, by using the application voltage line set as described above, it is possible to accurately detect the gas concentration (specifically, air/fuel ratio).

(2) In a second aspect which is a preferred embodiment of the gas concentration detection device (1), in a high-accuracy region in which high measurement accuracy for the air/fuel ratio is required, the gas concentration detection device is configured so as not to switch between the first voltage value and the second voltage value; and in a low-accuracy region in which a required measurement accuracy is lower than in the high-accuracy region, the gas concentration detection device is configured to switch between the first voltage value and the second voltage value.

Since the air/fuel ratio sensor (for example, zirconia oxygen sensor) has a capacitive component, as shown in FIG. 7, when voltage (Vp) is applied in a step form between the pair of electrodes, current (Ip) flowing between the electrodes changes in a spike form. Therefore, if voltage is applied in a step form in a high-accuracy region in which high measurement accuracy for the air/fuel ratio is required, there is a possibility that noise occurs in the current and the measurement accuracy for the air/fuel ratio is deteriorated.

Accordingly, in the second aspect, in the high-accuracy region in which high measurement accuracy is required, voltage switching is not carried out, and in the low-accuracy region in which required measurement accuracy is low, voltage switching is carried out.

Thus, it is possible to prevent the application voltage line from deviating from limiting current regions (which vary depending on the element temperature) while switching the voltage in a step form, and also to prevent deterioration in measurement accuracy in the high-accuracy region by carrying out voltage switching in the low-accuracy region.

The high-accuracy region is set so as to be shifted from the boundary between the first range and the second range, at which voltage switching is performed. For example, in the case of setting the high-accuracy region in the first range, the high-accuracy region is set inward of the lean-side boundary and the rich-side boundary of the first range, for example.

(3) In a third aspect which is a preferred embodiment of the gas concentration detection device (1) or (2) above, the gas concentration detection device is configured to switch between the first voltage value and the second voltage value based on the limiting current flowing between the pair of electrodes.

The third aspect exemplifies a preferred switching method for switching between the first voltage value and the second voltage value. The limiting current flowing between the pair of electrodes corresponds to the gas concentration (specifically, air/fuel ratio). Therefore, by switching between the first voltage value and the second voltage value in accordance with the limiting current, it is possible to set the application voltage line so that it is unlikely to deviate from the limiting current regions.

(4) In a fourth aspect which is a preferred embodiment of the gas concentration detection device of any of (1) to (3) above, the first range comprises a lean-side range indicating a predetermined air/fuel ratio range on a lean side from a stoichiometric state, and a rich-side range indicating a predetermined air/fuel ratio range on a rich side from the stoichiometric state.

The fourth aspect exemplifies a preferred range as the first range. For example, in the case where the target air/fuel ratio is set at a stoichiometric ratio, by setting the first range as described above, switching of the application voltage due to switching between the first range and the second range can be prevented in the vicinity of a stoichiometric state.

(5) In a fifth aspect which is a preferred embodiment of the gas concentration detection device (4) above, when the air/fuel ratio is on a lean side with respect to the lean-side range, the gas concentration detection device is configured to change the first voltage value to a lean-side second voltage value which is the second voltage value.

The fifth aspect exemplifies a voltage value set for switching the voltage on the lean side.

(6) In a sixth aspect which is a preferred embodiment of the gas concentration detection device of (4) or (5) above, when the air/fuel ratio is on a rich side with respect to the rich-side range, the gas concentration detection device is configured to change the first voltage value to a rich-side second voltage value which is the second voltage value.

The sixth aspect exemplifies a voltage value set for switching the voltage on the rich side.

<Hereinafter, Certain Configurations of the Present Invention Will Be Described>

The limiting current is, as is well known, a current value in a region (limiting current region) in which, even if voltage applied between the pair of electrodes changes, the value of current flowing between the pair of electrodes does not substantially change. The limiting current corresponds to a gas concentration (e.g., oxygen concentration or air/fuel ratio).

The air/fuel ratio is the mass ratio (A/F) of air (A) with respect to fuel (F). Here, a lean air/fuel ratio indicates that the amount of fuel is smaller than in a stoichiometric air/fuel ratio (stoichiometric state), and a rich air/fuel ratio indicates that the amount of fuel is larger than in a stoichiometric state.

The application voltage line defines the relationship between voltage applied between the pair of electrodes and current flowing through the pair of electrodes. The application voltage line is set so as to pass through a plurality of limiting current regions for a plurality of gas concentrations (specifically, air/fuel ratios).

Therefore, using the application voltage line, by, for example, setting current, the voltage applied between the pair of electrodes can be calculated.

### Effects of the Invention

The gas concentration detection device of the present invention enhances the detection accuracy in detecting the concentration of a specific gas in a gas to be measured.

### BRIEF DESCRIPTION OF THE DRAWINGS

FIG. 1 is an explanatory view which illustrates the system configuration of an air/fuel ratio sensor and a gas concentration detection device in an embodiment.

FIG. 2 is an explanatory cutaway view of a sensor element in the embodiment along the thickness direction (i.e., A-A cross-section in FIG. 3), as well as its electric configuration.

FIG. 3 is an explanatory partially-cutaway view of the sensor element in the embodiment, as seen from the thickness direction.

FIG. 4A is a graph which illustrates a basic relationship (V-I characteristics) between voltage and current of the air/fuel ratio sensor, and a limiting current region which changes in accordance with an air/fuel ratio.

FIG. 4B is a graph which illustrates a basic relationship (V-I characteristics) between voltage and current of the air/fuel ratio sensor, and a resistance-dominant region and a limiting current region which change in accordance with the element temperature.

FIG. 5 is a graph which illustrates an application voltage line set in the embodiment.

FIG. 6 is a flowchart which illustrates a control process for air/fuel ratio detection in the embodiment.

FIG. 7A is a graph which illustrates voltage applied in a step form.

FIG. 7B is a graph which illustrates a change in current in the case where a voltage in step form is applied.

### DESCRIPTION OF REFERENCE NUMERALS

Reference numerals used to identify various features in the drawings include the following.

5: air/fuel ratio sensor; 9: sensor element; 7: gas concentration detection device; 11: solid electrolyte layer; 21: measurement chamber; 23: reference oxygen chamber; 25: first electrode; 27: second electrode

### DETAILED DESCRIPTION OF THE PREFERRED EMBODIMENTS

Hereinafter, the present invention will be described in detail with reference to the drawings. However, the present invention should not be construed as being limited thereto.

In the following embodiments, a gas concentration detection device that measures gas concentration using an air/fuel ratio sensor which is a type of gas concentration sensor is described as an example.

### 1. Embodiment

### [1-1. Entire Configuration]

First, the entire configuration of a system relevant to a gas concentration detection device in an embodiment will be described.

As shown in FIG. 1, in the embodiment, for example, an air/fuel ratio sensor 5 is attached to an exhaust pipe 3 of an engine 1 of a vehicle, and a gas concentration detection device 7 detects an oxygen concentration (i.e., air/fuel ratio) in an exhaust gas discharged from the engine 1, based on output from the air/fuel ratio sensor 5.

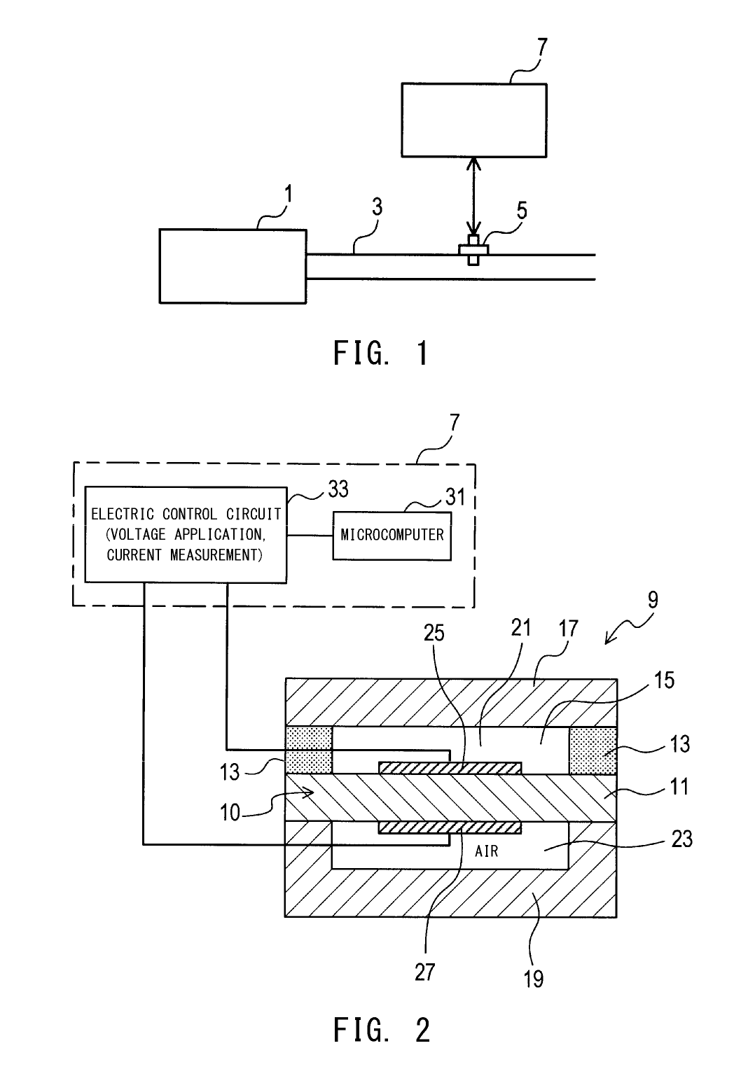

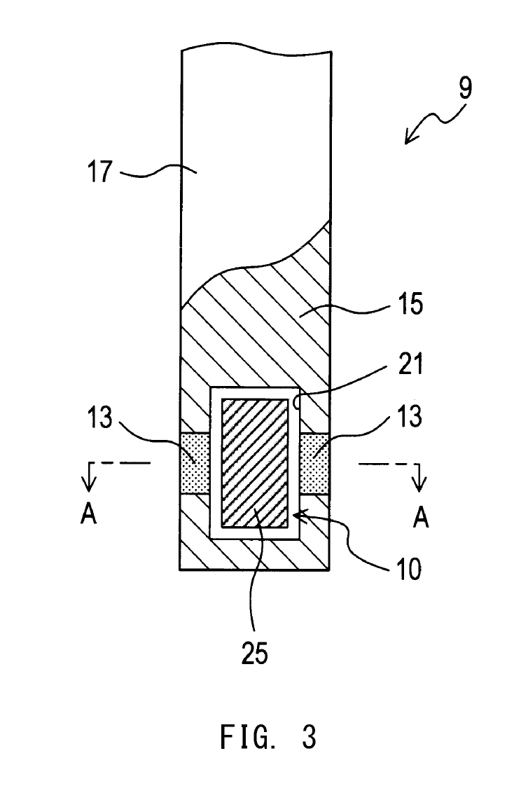

As shown in FIG. 2 and FIG. 3, the air/fuel ratio sensor 5 includes a stacked type sensor element 9 for detecting the oxygen concentration. The sensor element 9 is an elongated element and housed in a housing or the like (not shown).

More specifically, the sensor element 9 includes, in a layer form, a solid electrolyte (solid electrolyte layer) 11, a diffusion resistance layer 13, an intermediate insulating layer 15, a first outside insulating layer 17, and a second outside insulating layer 19, and includes a measurement chamber 21 and a reference oxygen chamber 23.

Among these, the solid electrolyte layer 11 is a rectangular plate material made from, for example, partially stabilized zirconia, and has a surface that faces the measurement chamber 21 and on which a first electrode 25 is formed, and a surface that faces the reference oxygen chamber 23 and on which a second electrode 27 is formed. That is, a pair of the first electrode 25 and the second electrode 27 are arranged so as to oppose each other with the solid electrolyte layer 11 therebetween. The electrodes 25, 27 are made from, for example, platinum. Here, the solid electrolyte layer 11 provided with the electrodes 25, 27 is referred to as an element portion 10.

The diffusion resistance layer 13 is a porous layer provided between the solid electrolyte layer 11 and the first outside insulating layer 17, and is made from alumina, zirconia, or the like, for example. An exhaust gas is introduced from outside (space in exhaust pipe 3) into the measurement chamber 21 through the diffusion resistance layer 13 so as to control diffusion of the exhaust gas.

The intermediate insulating layer 15 is a dense layer (layer formed so as not to allow gas penetration) provided between the solid electrolyte layer 11 and the first outside insulating layer 17, and is made from alumina, zirconia, or the like, for example. The intermediate insulating layer 15 together with the diffusion resistance layer 13 is provided so as to surround the periphery of the measurement chamber 21.

The first outside insulating layer 17 is a dense layer covering the measurement chamber 21, the intermediate insulating layer 15, and the diffusion resistance layer 13 from above in FIG. 2, and is made from alumina, zirconia, or the like, for example.

The second outside insulating layer 19 is a dense layer covering the periphery of the reference oxygen chamber 23, and is made from alumina, zirconia, or the like, for example. Although not shown, a heater for heating the sensor element 9 is embedded in the second outside insulating layer 19.

The measurement chamber 21 is a rectangular parallelepiped space into which an exhaust gas is introduced via the diffusion resistance layer 13 from the outside, and the first electrode 25 is formed on the solid electrolyte layer 11 inside the measurement chamber 21.

The reference oxygen chamber 23 is an elongated space into which air is introduced, and opens upward in FIG. 3. The second electrode 27 is formed on the solid electrolyte layer 11 inside the reference oxygen chamber 23.

Next, the electric configuration of the gas concentration detection device 7 will be described.

As shown in FIG. 2, the gas concentration detection device 7 is a device for controlling the operation of the sensor element 9 (i.e., the air/fuel ratio sensor 5) and detecting the oxygen concentration (i.e., the air/fuel ratio) in the exhaust gas, and includes an electric control circuit 33 and a microcomputer 31 programmed to direct the operation of the electronic control circuit 33.

The microcomputer 31 is an electronic control device including known CPU, ROM, RAM, and the like. The ROM stores data needed for control, such as an application voltage line, and rich-side switch current IpR and lean-side switch current IpL described below.

The electric control circuit 33 is a known circuit capable of applying a voltage (application voltage Vp) between the electrodes 25, 27 and measuring current (pump current Ip) flowing between the electrodes 25, 27, and is controlled by the microcomputer 31.

### [1-2. Basic Operation]

Next, pumping of oxygen, which is a basic operation of the air/fuel ratio sensor 5, will be described.

As shown in FIG. 2, in the sensor element 9, an ambient exhaust gas is introduced into the measurement chamber 21 via the diffusion resistance layer 13. Here, the case where a positive voltage is applied to the first electrode 25 and a negative voltage is applied to the second electrode 25 will be described.

First, in the case where the amount of fuel in the exhaust gas is such that the air/fuel ratio is greater than a stoichiometric state (stoichiometric air/fuel ratio: A/F=14.7) (a so-called lean case), oxygen in the exhaust gas is decomposed into oxygen ions at the first electrode 25 by applying a voltage (application voltage Vp) between the electrodes 25, 27.

The oxygen ions then pass through the solid electrolyte layer 11 from the first electrode 25, to be supplied to the second electrode 27, and then are discharged as oxygen from the second electrode 27 to the reference oxygen chamber 23. As a result, oxygen is pumped out of the measurement chamber 21. Thus, a current (pump current Ip) which is a positive current flows from the second electrode 27 side to the first electrode 25 side.

On the other hand, in the case where the amount of fuel in the exhaust gas is such that the air/fuel ratio is less than a stoichiometric state (a so-called rich case), opposite the lean case, oxygen in the reference oxygen chamber 23 is decomposed into oxygen ions at the second electrode 27.

Then, the decomposed oxygen ions pass through the solid electrolyte layer 11 from the second electrode 27, to be supplied to the first electrode 25, and then are discharged as oxygen from the first electrode 25 to the measurement chamber 21. As a result, oxygen is pumped into the measurement chamber 21. Thus, a current which is a negative current flows from the first electrode 25 side to the second electrode 27 side.

Therefore, as described in detail below, it is possible to set the application voltage Vp and detect the air/fuel ratio of the exhaust gas, based on the pump current Ip described above.

### [1-3. Relationship Between Voltage and Current]

Next, the relationship between the application voltage Vp and the pump current Ip, and the application voltage line ID used in gas concentration detection will be described.

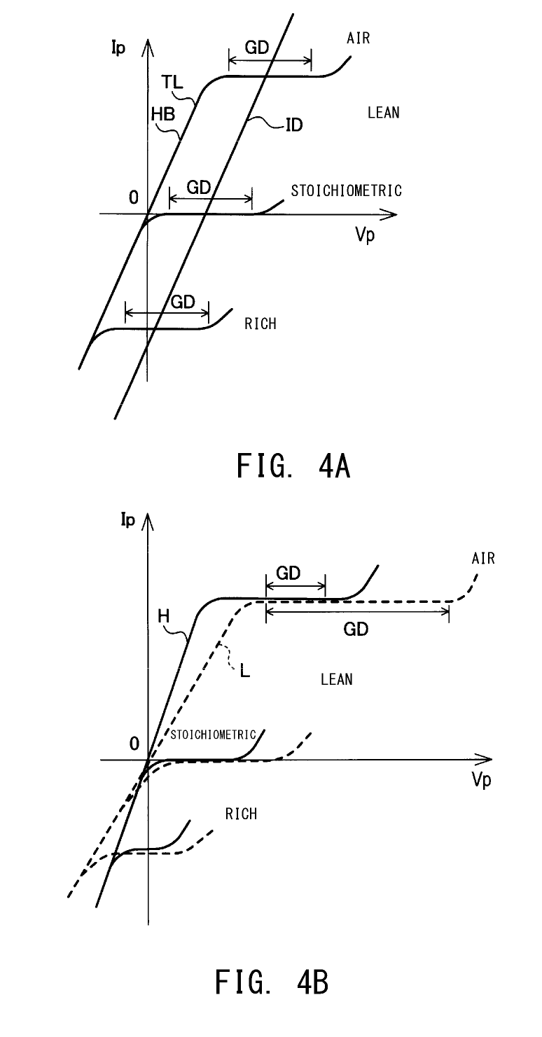

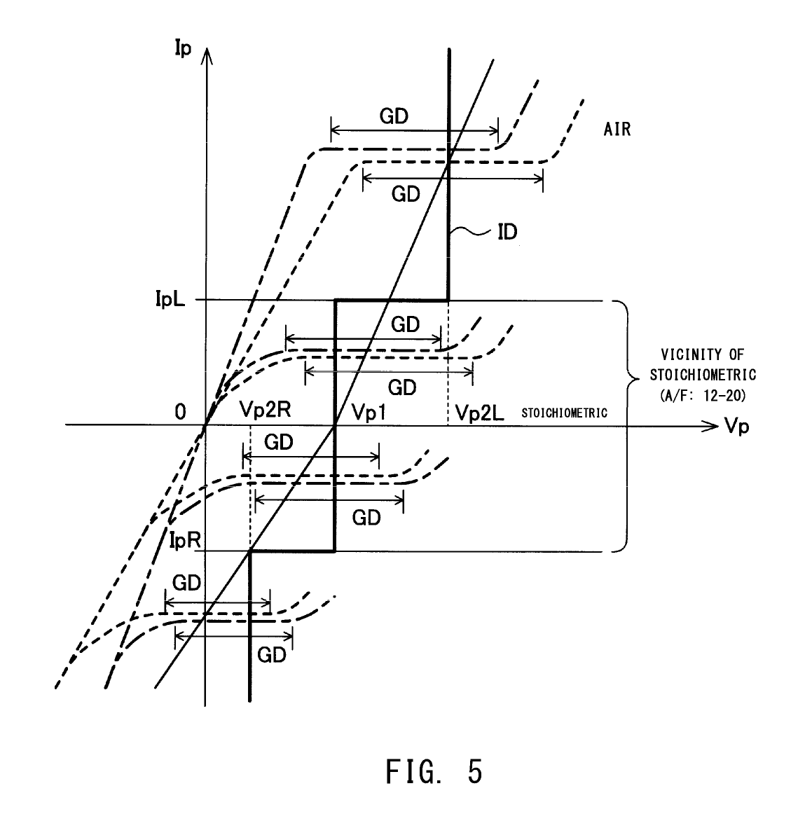

As shown in FIG. 4A, a graph (characteristics line TL) indicating the relationship between the application voltage Vp and the pump current Ip has a proportional part HB in which the pump current Ip changes in proportion to an increase in the application voltage Vp, and a flat part parallel to the voltage axis.

Of these parts, the proportional part HB is a resistance-dominant region influenced by a DC (direct current) internal resistance Ri (hereinafter, also referred to as a resistance R) of the element portion 10 (specifically, the solid electrolyte layer 11) of the sensor element 9. That is, in the resistance dominant region, as the application voltage Vp increases, the pump current Ip increases in proportion thereto. The resistance R changes in accordance with the temperature (element temperature) of the sensor element 9 (specifically, the solid electrolyte layer 11), as described below.

The flat part is a part in which, even if the application voltage Vp changes, the pump current Ip does not substantially change but remains at a constant value (limiting current). The flat part is a limiting current region GD which indicates the pump current Ip corresponding to the oxygen concentration (i.e., air/fuel ratio), and in which a change in the limiting current corresponds to a change in the air/fuel ratio.

That is, as the air/fuel ratio approaches the lean side, the limiting current of the pump current Ip increases, and as the air/fuel ratio approaches the rich side, the limiting current decreases. Therefore, the air/fuel ratio can be calculated from the limiting current.

For example, assuming a simple characteristics line TL as shown in FIG. 4A, a single straight application voltage line ID indicating application voltage characteristics may be set so as to pass through limiting current regions GD according to the respective air/fuel ratios, and the air/fuel ratio may be detected using the application voltage line ID. That is, a predetermined voltage in accordance with the application voltage line ID may be applied and the air/fuel ratio may be calculated based on the pump current Ip (indicating the limiting current) obtained at that time.

However, as described above, in the characteristics line TL, the low-voltage side (the left side in FIG. 4A) with respect to the limiting current region GD is a resistance-dominant region influenced by the resistance R of the solid electrolyte layer 11, and has characteristics which change in accordance with the element temperature.

Specifically, as shown in FIG. 4B, if the element temperature decreases (in case of a low-temperature side L), the resistance R increases and the slope of the straight proportional part HB decreases. On the other hand, if the element temperature increases (in case of a high-temperature side H on which the temperature is higher than on the low-temperature side L), the resistance R decreases and the slope of the straight proportional part HB increases.

In addition, when the element temperature changes as described above, as shown in FIG. 4B, not only the slope (slope of proportional part HB) of the characteristics line TL but also the limiting current region GD changes along the direction of the voltage axis (see, for example, limiting currents GD on high-temperature side H and low-temperature side L in the air).

Therefore, it is necessary to also set the application voltage line ID in consideration of a change in the limiting current region GD due to a change in the element temperature.

Accordingly, in the present embodiment, as shown in FIG. 5, a single application voltage line ID is set so as to change stepwise at predetermined values (i.e., at predetermined pump currents Ip) on the lean side and the rich side.

Specifically, in the application voltage line ID, the application voltage Vp is set at a first voltage value (for example, 450 [mV]) which is a fixed value, in a predetermined range (first range) straddling a stoichiometric state between the lean side and the rich side.

That is, the application voltage Vp is set at a first voltage value Vp1, when the pump current Ip (that is limiting current) is in a range from a rich-side switch current IpR to a lean-side switch current IpL, which corresponds to an air/fuel ratio (A/F) range of 12 to 20.

The range from the rich-side switch current IpR to the lean-side switch current IpL (here, IpL>IpR) is the first range. In addition, the range of the pump current Ip from the lean-side switch current IpL to 0 is a lean-side range in the first range, and the range of the pump current Ip from the rich-side switch current IpR to 0 is a rich-side range in the first range.

Further, in the application voltage line ID, in a predetermined range (lean-side second range) higher than the lean-side switch current IpL, the application voltage Vp is set at a lean-side second voltage value Vp2L (for example, 700 [mV]) which is a fixed value.

On the other hand, in the application voltage line ID, in a predetermined range (rich-side second range) lower than the rich-side switch current IpR, the application voltage Vp is set at a rich-side second voltage value Vp2R (for example, 300 [mV]) which is a fixed value.

Thus, the application voltage line ID is set so as to switch among the first voltage value Vp1, the lean-side second voltage value Vp2L, and the rich-side second voltage value Vp2R when the pump current Ip becomes the lean-side switch current IpL or the rich-side switch current IpR.

Specifically, when the pump current Ip becomes equal to or greater than the rich-side switch current IpR from a value smaller than the rich-side switch current IpR, the pump voltage Vp is switched from the rich-side second voltage value Vp2R to the first voltage value Vp1.

Conversely, when the pump current Ip becomes smaller than the rich-side switch current IpR from a value equal to or greater than the rich-side switch current IpR, the pump voltage Vp is switched from the first voltage value Vp1 to the rich-side second voltage value Vp2R.

On the other hand, when the pump current Ip becomes equal to or smaller than the lean-side switch current IpL from a value greater than the lean-side switch current IpL, the pump voltage Vp is switched from the lean-side second voltage value Vp2L to the first voltage value Vp1.

Conversely, when the pump current Ip becomes greater than the lean-side switch current IpL from a value equal to or smaller than the lean-side switch current IpL, the pump voltage Vp is switched from the first voltage value Vp1 to the lean-side second voltage value Vp2L.

The first range is a range (for example, A/F range of 12 to 20) corresponding to a range from the lean-side switch current IpL to the rich-side switch current IpR. In addition, the lean-side second range is a range (for example, A/F range higher than 20) corresponding to a value higher than the lean-side switch current IpL, and the rich-side second range is a range (for example, A/F range lower than 12) corresponding to a value lower than the rich-side switch current IpR. That is, in the present embodiment, the air/fuel ratio range corresponding to a gas concentration measurement range is divided into three ranges.

In addition, a high-accuracy region is set inside the first range so as not to overlap the boundary between the first range and the second range. Specifically, the high-accuracy region is set to be a range below the lean-side switch current IpL and above the rich-side switch current IpR so as to include a stoichiometric state, for example, an A/F range of 13 to 16. Here, a region other than the high-accuracy region is a low-accuracy region.

### [1-4. Control]

Next, a process for detecting the oxygen concentration (air/fuel ratio) using the application voltage line ID by the microcomputer 31 will be described.

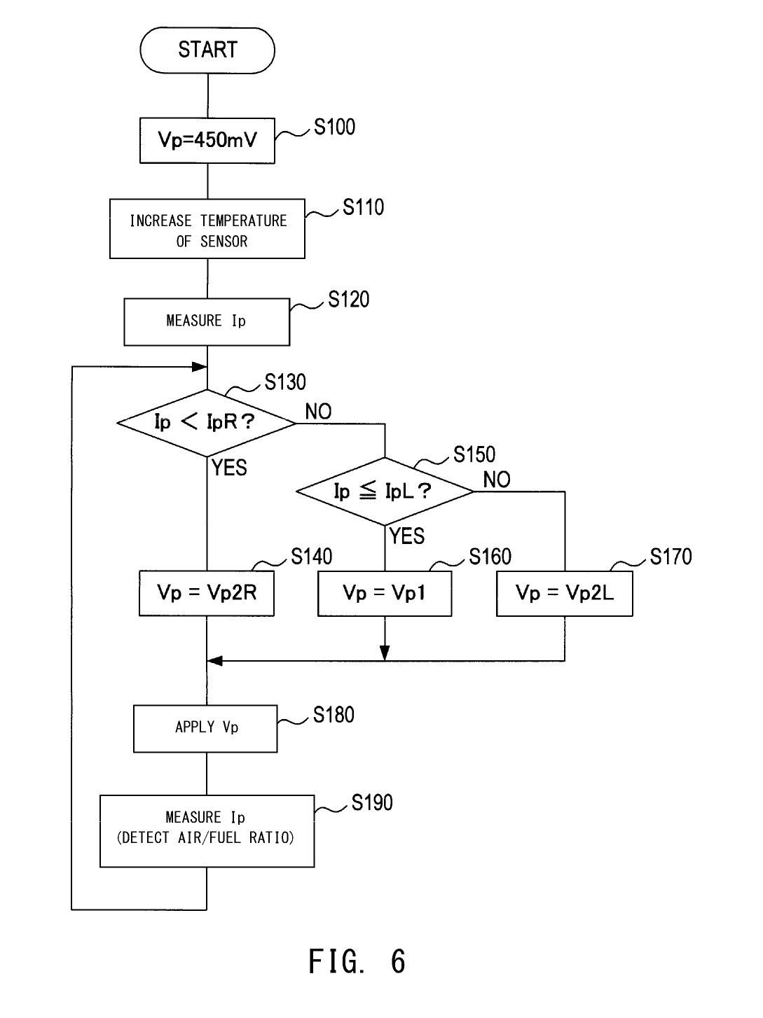

As shown in FIG. 6, first, in step (S) 100, 450 [mV] is set as an initial value of the application voltage Vp.

In the subsequent step 110, control is performed to increase the temperature (element temperature) of the sensor element 9 by applying a voltage to the heater. Thereafter, as is well known to those of ordinary skill in this field of art, the heater is controlled so as to keep the element temperature at a target temperature.

In the subsequent step 120, at the target temperature, voltage of 450[mV] set in step 100 is applied between the pair of electrodes 25, 27, using the electric control circuit 33, and the pump current Ip flowing between the pair of electrodes 25, 27 at that time is measured.

In the subsequent step 130, a determination is made as to whether or not the pump current Ip measured in step 120 is smaller than the rich-side switch current IpR. If the determination result is positive, the process proceeds to step 140, and on the other hand, if the determination result is negative, the process proceeds to step 150.

In step 140, since the pump current Ip is smaller than the rich-side switch current IpR, the air/fuel ratio is considered to be in the rich-side second range, and the application voltage Vp is set at the rich-side second voltage value Vp2R.

On the other hand, in step 150, a determination is made as to whether or not the pump current Ip is equal to or smaller than the lean-side switch current IpL. If the determination result is positive, the process proceeds to step 160, and on the other hand, if the determination result is negative, the process proceeds to step 170.

In step 160, since the pump current Ip is equal to or greater than the rich-side switch current IpR (according to the determination result in step 130) and is equal to or smaller than the lean-side switch current IpL (according to the determination result in step 150), the air/fuel ratio is considered to be in the first range, and the application voltage Vp is set at the first voltage value Vp1.

On the other hand, in step 170, since the pump current Ip is greater than the lean-side switch current IpL, the air/fuel ratio is considered to be in the lean-side second range, and the application voltage Vp is set at the lean-side second voltage value Vp2L.

In step 180 subsequent to steps 140, 160, 170, the application voltage Vp set in each step 140, 160, 170 is applied between the pair of electrodes 25, 27.

In the subsequent step 190, the pump current Ip flowing between the pair of electrodes 25, 27 by voltage applied in step 180 is measured, and then the process returns to step 130.

Since the measured pump current Ip corresponds to the oxygen concentration, the oxygen concentration can be calculated from the pump current Ip. Since the oxygen concentration corresponds to the air/fuel ratio, the air/fuel ratio can be calculated from the pump current Ip, using a map or the like.

### [1-5. Effects]

In the present embodiment, as a basic configuration, the application voltage line ID is set so as to pass through the respective limiting current regions GD for different oxygen concentrations (i.e., air/fuel ratios), and to pass through a region in which the respective limiting current regions GD for different temperature conditions of the sensor element 9 (specifically, element portion 10) overlap each other.

In addition, in the present embodiment, in the basic configuration described above, as the voltage set by the application voltage line ID, an identical first voltage value Vp1 is used when the air/fuel ratio corresponding to the gas concentration is in the predetermined first range, and an identical lean-side second voltage value Vp2 (different from first voltage value Vp1) is used when the air/fuel ratio is in the lean-side second range adjacent to the first range. Similarly, an identical lean-side second voltage value Vp2 (different from first voltage value Vp1) is used when the air/fuel ratio is in the rich-side second range adjacent to the first range, and the first voltage value and the second voltage value are switched (in a step form) between the first range and the second range. Thus, the gas concentration (specifically, air/fuel ratio) can be accurately detected.

That is, when considering the temperature condition, for example, depending on the oxygen concentration or the like, the application voltage line using a single straight line can deviate from some limiting current regions, and in this case, it might be impossible to accurately detect, for example, the oxygen concentration even if control using this application voltage line is performed.

However, in the present embodiment, the first voltage value and the second voltage value are switched between the first range and the second range, and in addition, the first voltage value Vp which is an identical fixed value is used in the first range, the second voltage value Vp2L which is an identical fixed value is used in the lean-side second range, and the second voltage value Vp2R which is an identical fixed value is used in the rich-side second range. Thus, the application voltage line ID can be prevented from deviating from the limiting current regions GD. Therefore, by using the application voltage line ID set as described above, it is possible to accurately detect the gas concentration (specifically, air/fuel ratio).

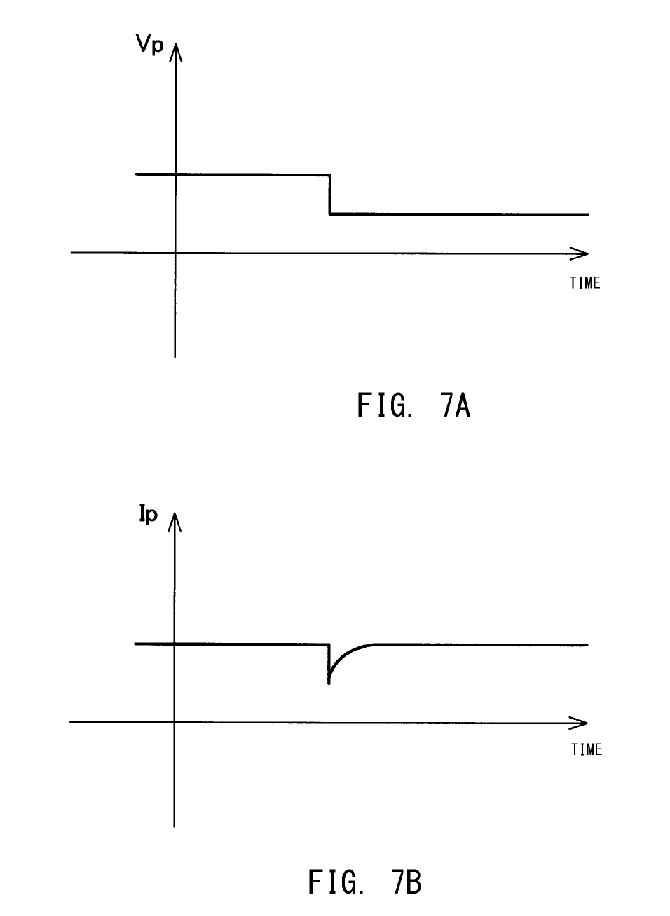

In addition, in the present embodiment, in the high-accuracy region in which high measurement accuracy is required, the application voltage Vp is not switched, and in the low-accuracy region in which required measurement accuracy is low, the application voltage Vp is switched in a step form as shown in FIG. 7A, for example.

As a result, as shown in FIG. 7B, the pump current Ip changes in a spike form, but this region is not the high-accuracy region and therefore there is no adverse effect on the high measurement accuracy. Thus, it is possible to ensure high measurement accuracy in the high-accuracy region.

Further, in the present embodiment, switching between the first voltage value Vp1 and the second voltage value Vp2 (i.e., Vp2L, Vp2R) is performed based on the limiting current flowing between the pair of electrodes 25, 27.

That is, the limiting current flowing between the pair of electrodes 25, 27 corresponds to the gas concentration (specifically, air/fuel ratio), and therefore, by switching between the first voltage value Vp1 and the second voltage value Vp2 in accordance with the limiting current, it is possible to easily set the application voltage line ID that is unlikely to deviate from the limiting current regions GD.

### [1-6. Term Correspondence]

Here, the relationship between corresponding terms used to define the invention and the first embodiment will be described.

The solid electrolyte layer 11, the electrodes 25, 27, the air/fuel ratio sensor 5, and the gas concentration detection device 7 in the present embodiment correspond to examples of a solid electrolyte, electrodes, a gas concentration sensor, and a gas concentration detection device in the present invention, respectively.

### 2. Other Embodiments

While certain embodiments of the present invention have been described above, the present invention is not limited thereto, but may be modified in various ways without deviating from the gist of the invention.

(1) For example, in the above embodiment, a predetermined fixed value (450 [mV]) is used, but another value may be used.

(2) In the above embodiments, a gas concentration detection device that detects an oxygen concentration using an oxygen sensor (air/fuel ratio sensor) for detecting the oxygen concentration has been shown. However, the present invention is also applicable to a gas concentration detection device that detects a gas concentration of NOₓ, H₂O, or the like, for example.

(3) The components in the above embodiments may be combined as appropriate.

The invention has been described in detail with reference to the above embodiments. However, the invention should not be construed as being limited thereto. It should further be apparent to those skilled in the art that various changes in form and detail of the invention as shown and described above may be made. It is intended that such changes be included within the spirit and scope of the claims appended hereto.

This application is based on Japanese Patent Application No. 2016-100624 filed May 19, 2016, incorporated herein by reference in its entirety.

What is claimed is:

1. A gas concentration detection device adapted for operating a gas concentration sensor including a sensor element having: a solid electrolyte having oxygen ion conductivity; and a pair of electrodes formed on the solid electrolyte, the gas concentration detection device being configured to apply a voltage between the pair of electrodes based on an application voltage line which defines a relationship between voltage applied between the pair of electrodes and current flowing between the pair of electrodes, detect a limiting current flowing between the pair of electrodes in accordance with the voltage, and detect a gas concentration of a specific component in a gas to be measured, based on the limiting current, wherein
in a detection range for detecting the gas concentration, the application voltage line is set so as to pass through a plurality of limiting current regions of: respective limiting current regions for different values of the gas concentration; and a region in which respective limiting current regions for different temperature conditions of the sensor element overlap each other, and
the voltage set by the application voltage line is an identical first voltage value used when an air/fuel ratio corresponding to the gas concentration is in a predetermined first range, is an identical second voltage value different from the first voltage value used when the air/fuel ratio is in a second range adjacent to the first range, and the first voltage value and the second voltage value are switched between the first range and the second range.

2. The gas concentration detection device as claimed in claim 1, wherein
in a high-accuracy region in which high measurement accuracy for the air/fuel ratio is required, the gas concentration detection device is configured so as not to switch between the first voltage value and the second voltage value, and in a low-accuracy region in which a required measurement accuracy is lower than in the high-accuracy region, the gas concentration detection device is configured to switch between the first voltage value and the second voltage value.

3. The gas concentration detection device as claimed in claim 1, wherein the gas concentration detection device is configured to switch between the first voltage value and the second voltage value based on the limiting current flowing between the pair of electrodes.

4. The gas concentration detection device as claimed in claim 1, wherein
the first range comprises a lean-side range indicating a predetermined air/fuel ratio range on a lean side from a stoichiometric state, and a rich-side range indicating a predetermined air/fuel ratio range on a rich side from the stoichiometric state.

5. The gas concentration detection device as claimed in claim 4, wherein
when the air/fuel ratio is on a lean side with respect to the lean-side range, the gas concentration detection device is configured to change the first voltage value to a lean-side second voltage value which is the second voltage value.

6. The gas concentration detection device as claimed in claim 4, wherein
when the air/fuel ratio is on a rich side with respect to the rich-side range, the gas concentration detection device is configured to change the first voltage value to a rich-side second voltage value which is the second voltage value.

7. The gas concentration detection device as claimed in claim 5, wherein
when the air/fuel ratio is on a rich side with respect to the rich-side range, the gas concentration detection device is configured to change the first voltage value to a rich-side second voltage value which is the second voltage value.
